(19) United States
(12) Plant Patent Application Publication
(10) Pub. No.: US 2002/0073463 P1
(43) Pub. Date: Jun. 13, 2002

(54) VARIETY OF GERANIUM PLANT NAMED 'TIKPINK'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,024

(22) Filed: Dec. 8, 2000

(51) Int. Cl. A01H 5/00

(52) U.S. Cl. PLT/328

(57) ABSTRACT

A new and distinct zonal geranium plant with intense pink colored flowers above medium green foliage. The plant flower is suitable for solitaire pot planting.

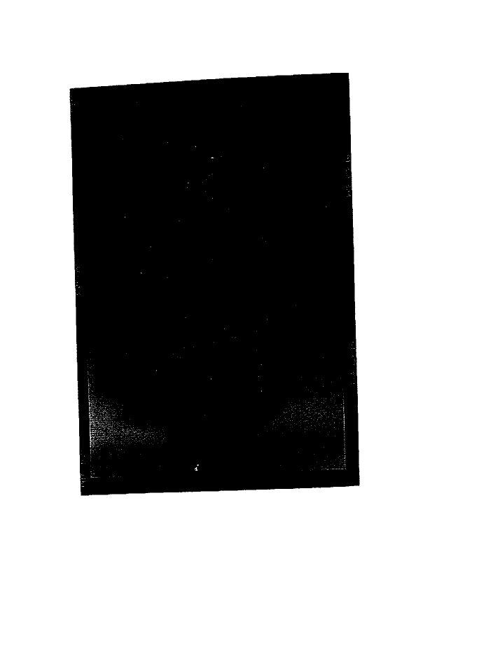

### BACKGROUND OF THE INVENTION

The present invention comprises a new and distinct cultivar of Pelargonium x hortorum known by the varietal name ‘Tikpink’. The new variety was discovered in 1996 in a selective breeding program in Dresden, Germany. The new variety is the result of a combination breeding of 60 seedlings having pink colored flowers. The new variety was first asexually reproduced by cuttings in Dresden, Germany in 1997. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to The R.H.S. Colour Chart of The Royal Horticultural Society of London.

Plant:

Classification.—Botanical: Pelargonium x hortorum. Commercial: Zonal geranium.

Form.—Erect.

Overall height.—120 m after 1 year.

Height from media surface to top of foliage.—70 cm after 6 months.

Height from media surface to top of umbel.—65-70 cm.

Growth.—Erect.

Strength.—Requires support.

Response time.—4 weeks.

Leaves.—Size: 6.0-8.0 cm long. Shape: Cordate. Margin: Emarginate. Texture: Hairy. Color: Green Group 137A with prominent zone. Ribs and veins: Color: Brighter than leaf surface.

Petioles.—Color: Medium green. Length: 7.0-9.0 cm.

Stem/branches.—Color: Medium green. Internode length: 3.0-5.0 cm.

The bud:

Shape when just showing color.—Overall: Semi-spherical. Individual bud: Narrow; elliptical.

Color.—Yellow-Green Group 144A.

Size when just showing color.—Umbel: 8.0-10.0 cm across. Individual bud: 1.5 cm long; 1.0 cm wide.

Inflorescence:

Blooming habit.—Single.

Size of fully open bloom.—4.0-5.0 cm across.

Borne.—Flowers on umbel; umbel on pedicel; pedicel on peduncle.

Flowers.—Form: Zygomorph. Number of petals: 5. Size: 2.0 cm wide; 2.5 cm long. Petals: Color: Red-Purple Group 66C. Texture and appearance: Velvety.

Petaloids.—Absent.

Pedicel.—Length: 2.5-3.0 cm. Color: Medium green.

Peduncle.—Length: 11.0-13.0cm. Color: Medium green.

Disease/pest resistance.—No unusual susceptibility to diseases or pests has been noted to date.

Lasting Quality.—Semi-long-lasting; 2 weeks per umbel.

Reproductive organs:

Stamens.—Anthers: 3 mm long. Filaments: Length: 0.5 cm maximum. Color: Pink. Pollen: Yellow.

Pistils.—Number: Five. Length: 0.3-0.4 cm. Stigma: Pink. Style: 0.8 cm long.

Ovaries.—Color: Greenish. Length: 0.4 cm.

Fruit.—Middle.

### GENERAL CHARACTERISTICS

1. Suitable for solitaire pot plant.

2. Must be bound to get a pyramid shape.

3. Pinched several times for groundcover.

4. Very decorative.

5. Long-lasting.

I claim:

1. A new and distinct variety of geranium plant substantially as shown and described.
